(19) United States
(12) Patent Application Publication
(10) Pub. No.: US 2021/0330755 A1
(43) Pub. Date: Oct. 28, 2021

(54) HALOPEROXIDASE COMPOSITIONS AND USES THEREOF

(71) Applicant: Exoxemis, Inc., Little Rock, AR (US)

(72) Inventors: Robert C. Allen, Omaha, AR (US); Jackson T. Stephens, Little Rock, AR (US)

(21) Appl. No.: 17/259,644

(22) PCT Filed: Jul. 11, 2019
(86) PCT No.: PCT/US2019/041419

§ 371 Date: Jan. 12, 2021

Related U.S. Application Data
(60) Provisional application No. 62/697,828, filed on Jul. 13, 2018.

(51) Int. Cl. A61K 38/44; A61P 7/00

(52) U.S. Cl. CPC A61K 38/44; C12Y 111/01007; C12Y 111/02002; A61P 7/00

(57) ABSTRACT

A method of treating at least one of septicemia, endotoxemia or a bacterial infection in a subject can include administering to the subject a composition containing a therapeutically effective amount of a haloperoxidase. The haloperoxidase can be myeloperoxidase or eosinophil peroxidase. The haloperoxidase can inhibit lipopolysaccharide or Lipid A activity in the subject; as a non-limiting example, the haloperoxidase is not engaged in active haloperoxidase activity, but available for LPS or Lipid A binding and inhibition.

### FIELD OF THE PRESENT DISCLOSURE

The present disclosure relates to compositions comprising a haloperoxidase, and the use of such compositions in the treatment of bacterial infections, particularly Gram-negative bacterial infections, and endotoxin-related disorders.

### BACKGROUND

Any discussion of the prior art throughout the specification should in no way be considered as an admission that such prior art is widely known or forms part of the common general knowledge in the field.

Bacterial toxins promote infection and disease by directly damaging host tissues and by modulating the host's immune system. There are two types of bacterial toxins, namely exotoxins and endotoxins. Exotoxins are secreted by bacteria and cause a variety of symptoms which will depend on the species of bacteria from which the exotoxins are secreted. Endotoxins, also known as lipopolysaccharides (LPS), are a potent stimulator of host immune systems. They comprise a toxic lipid component known as Lipid A as well as O antigen (or O polysaccharide) and a core oligosaccharide. Endotoxins are found in the outer membrane of Gram-negative bacteria where they provide structural integrity and protect the membrane from chemical attack. When bacterial cells are lysed by the immune system or antibacterial agents, fragments of the membrane, including LPS, may be released into the circulation, where they can cause septicemia and severe endotoxemia as well as a range of related conditions such as sepsis, fever, diarrhea, septic shock, and loss of function of internal organs such as kidneys, liver, adrenal glands, and lungs.

Gram-negative bacterial infections are challenging to treat because bacterial death often leads to the release of endotoxins. For example, the Jarisch-Herxheimer reaction, a condition with signs and symptoms resembling bacterial sepsis, occurs when endotoxin is released from Gram-negative spirochete bacteria, such as Treponema (the causative agent of syphilis) and Borrelia (the causative agent of Lyme's disease) during antibiotic treatment.

In this context, there is a need for compositions and methods for treating bacterial infections and endotoxin-related disorders.

### SUMMARY

In one aspect, the present disclosure provides a method of treating septicemia in a subject, the method comprising administering to the subject a composition comprising a therapeutically effective amount of a haloperoxidase.

The haloperoxidase may be a myeloperoxidase or eosinophil peroxidase.

In certain embodiments, the composition is administered systemically to the subject. The composition may be administered parenterally to the subject. Preferably, the composition is administered intravenously to the subject.

The subject is preferably a human.

The haloperoxidase may inhibit lipopolysaccharide or Lipid A activity in the subject.

In certain embodiments, the haloperoxidase is not engaged in active haloperoxidase activity, but available for LPS or Lipid A binding and inhibition.

In certain embodiments, the composition comprises the haloperoxidase at a concentration of less than about 50,000 μg/mL. Preferably, the composition comprises the haloperoxidase at a concentration of less than about 1,000 μg/mL.

The haloperoxidase is preferably myeloperoxidase.

In certain embodiments, the septicemia is associated with Gram-negative sepsis, organ dysfunction, acute respiratory distress syndrome, systemic inflammatory response syndrome or septic shock.

In another aspect, the present disclosure provides a method of treating endotoxemia in a subject, the method comprising administering to the subject a composition comprising a therapeutically effective amount of a haloperoxidase.

The haloperoxidase may be myeloperoxidase or eosinophil peroxidase.

In certain embodiments, the composition is administered systemically to the subject. The composition may be administered parenterally to the subject. Preferably, the composition is administered intravenously to the subject.

In certain embodiments, the endotoxemia is associated with a concentration of endotoxin in serum of the subject of greater than about 5 pg/mL. The endotoxemia may be associated with a concentration of endotoxin in serum of the subject of greater than about 20 pg/mL.

The subject is preferably a human.

In certain embodiments, the haloperoxidase inhibits lipopolysaccharide or Lipid A activity in the subject.

In certain embodiments, the haloperoxidase is not engaged in active haloperoxidase activity, but available for LPS or Lipid A binding and inhibition.

In certain embodiments, the composition comprises the haloperoxidase at a concentration of less than about 50,000 μg/mL. Preferably, the composition comprises the haloperoxidase at a concentration of less than about 1,000 μg/mL.

The haloperoxidase is preferably myeloperoxidase.

In certain embodiments, the endotoxemia is associated with septicemia, sepsis, toxic shock, fever, diarrhea, septic shock, gastroenteritis, pneumonia, meningitis, endocarditis, osteomyelitis, cholecystitis, cholangitis, meningitis, enteric fever, shigellosis, multiple organ dysfunction syndrome, peritonitis, neutropenia, urosepsis, liver dysfunction, kidney dysfunction, pancreatitis, leaky bowel syndrome, menigingococcemia or systemic inflammatory response syndrome. In particular embodiments, the endotoxemia is associated with septicemia.

In a further aspect, the present disclosure provides a method of treating a bacterial infection in a subject, the method comprising administering to the subject a composition comprising a therapeutically effective amount of a haloperoxidase.

The haloperoxidase may be myeloperoxidase or eosinophil peroxidase.

In certain embodiments, the composition is administered systemically to the subject. The composition may be administered parenterally to the subject. Preferably, the composition is administered intravenously to the subject.

The subject is preferably a human.

In certain embodiments, the bacterial infection is a Gram-negative bacterial infection.

The haloperoxidase may inhibit lipopolysaccharide or Lipid A activity in the subject.

In certain embodiments, the haloperoxidase is not engaged in active haloperoxidase activity, but available for LPS or Lipid A binding and inhibition.

In certain embodiments, the composition comprises the haloperoxidase at a concentration of less than about 50,000 μg/mL. Preferably, the composition comprises the haloperoxidase at a concentration of less than about 1,000 μg/mL.

The haloperoxidase is preferably myeloperoxidase.

In certain embodiments, the bacterial infection is caused by Treponema spp., Borrelia spp., Citrobacter freundii, Citrobacter sedlakii, Citrobacter braakii, Citrobacter werkmanii, Citrobacter youngae, Citrobacter amalonaticus, Enterobacter cloacae, Enterobacter cancerogenus, Enterobacter hormaechei, Enterobacter asburiae, Escherichia coli, Escherichia fergusonii, Klebsiella pneumoniae, Proteus mirabilis, Acinetobacter spp., Pseudomonas aeruginosa, Aeromonas hydrophila, Pasteurella multocida, Shigella boydii, Shigella dysenteriae, Shigella flexneri, Shigella sonnei, Salmonella choleraesuis, Salmonella choleraesuis subsp. indica, Salmonella enteritidis, Salmonella virchow, Salmonella paratyphi B, Salmonella typhimurium, Salmonella paratyphi A, Salmonella typhi, Salmonella choleraesuis subsp. arizonae, Salmonella choleraesuis subsp. diarizonae, Salmonella choleraesuis subsp. houtenae, Salmonella bongori, Yersinia enterocolitica, Yersinia frederiksenii, Yersinia pestis or Yersinia pseudotuberculosis.

In another aspect, the present disclosure provides a composition suitable for treating a bacterial infection in a subject, the composition comprising: a therapeutically effective amount of a haloperoxidase; and a pharmaceutically acceptable carrier, wherein the composition is suitable for systemic administration to the subject.

The haloperoxidase may be myeloperoxidase or eosinophil peroxidase.

In certain embodiments, the composition is suitable for parenteral administration. Preferably, the composition is suitable for intravenous administration and/or topical application.

In certain embodiments, the bacterial infection is a Gram-negative bacterial infection.

In certain embodiments, the haloperoxidase is not engaged in active haloperoxidase activity, but available for LPS or Lipid A binding and inhibition.

In certain embodiments, the composition comprises the haloperoxidase at a concentration of less than about 50,000 μg/mL. Preferably, the composition comprises the haloperoxidase at a concentration of less than about 1,000 μg/mL.

The subject is preferably a human.

Preferably, the haloperoxidase is myeloperoxidase.

In another aspect, the present disclosure provides a method of treating a Gram-negative bacterial infection in a subject, the method comprising administering to the site of the Gram-negative bacterial infection in the subject a composition comprising: a therapeutically effective amount of a haloperoxidase; wherein the haloperoxidase is selected from myeloperoxidase and eosinophil peroxidase.

The haloperoxidase may inhibit lipopolysaccharide or Lipid A activity in the subject.

In certain embodiments, the composition comprises from about 1 μg/mL to about 50,000 μg/mL of the haloperoxidase.

In certain embodiments, the composition comprises from 10 to 5,000 μg/mL of the haloperoxidase.

In certain embodiments, the subject is suffering from a bacterial infection of the gum, eye, ear, skin, soft tissue, wound, vaginal area, groin area, bed sore or burn area, and preferably the method is a non-systemic use for treatment of superficial infection or wound.

The haloperoxidase is preferably eosinophil peroxidase.

The subject is preferably a human.

In another aspect, the present disclosure provides a method of treating a Gram-negative bacterial infection in a subject the method comprising administering to the site of the Gram-negative bacterial infection in the subject a first composition comprising a therapeutically effective amount of a haloperoxidase, wherein the haloperoxidase inhibits lipopolysaccharide or Lipid A activity; and administering to the site of the bacterial infection in the subject a second composition comprising a substrate for the haloperoxidase; wherein the haloperoxidases of the first composition generate microbicidal agents, such as hypochlorite and singlet molecular oxygen, when combined with the second composition (i.e., the substrate) to kill Gram-negative bacteria as well as, Gram-positive bacteria, yeast and fungi that may be present.

The haloperoxidase is preferably myeloperoxidase or eosinophil peroxidase.

In certain embodiments, the first composition and the second composition are mixed before administration to the site of infection.

In certain embodiments, the first composition and the second composition are administered concurrently to the site of infection.

In certain embodiments, the first composition and the second composition are administered sequentially to the site of infection.

The haloperoxidase is preferably eosinophil peroxidase.

The subject is preferably a human.

### BRIEF DESCRIPTION OF THE DRAWINGS

FIG. 1. A regression plot of lipopolysaccharide (LPS) standards (ng/well) against time (seconds) to maximum slope. Derived equation and coefficients of determination (R²) are shown for two ranges: 0.0012 ng to 5 ng LPS and 0.0012 ng to 0.625 ng LPS. Standards testing was performed in duplicate.

FIG. 2. Percent inhibition of Limulus amebocyte lysate (LAL) assay activity measured at 5.0 ng/well LPS and plotted against different amounts (mg/well) of myeloperoxidase (MPO). The experiment was run in quadruplicate with all data points depicted. The regression equation is shown with the R² fit.

FIG. 3. Percent inhibition of LAL activity using 0.1 ng LPS/well plotted against mg/well of MPO (upper curve), eosinophil peroxidase (EPO) (middle curve) and glucose oxidase (GO) (lower curve) with derived equation and R² fit.

FIG. 4. A regression plot of Lipid A standards against time (seconds) to maximum slope with derived equations and R² fit. The four curves were obtained at 2.5, 3.75, 4.5 and 5.75 hours after preparation of the Lipid A standards. The order of the curves, starting from the upper most curve and working downwards, is 5.75 hours, 4.5 hours, 3.75 hours and 2.5 hours.

FIG. 5. Percent inhibition of chromogenic LAL activity measured at 2.16 μg Lipid A/well plotted against MPO or EPO in mg/well. The derived inhibition equations and R² fits are shown.

FIG. 6. Percent inhibition of LPS endotoxin activities by a composition comprising 4 μg MPO and 0.8 μg GO/well measured using the chromogenic LAL assay. The composition was tested without D-glucose (upper, dashed curve) and with a non-limiting concentration (0.2 mg/well) of D-glucose (lower curve).

FIG. 7. Kaplan Meier survivor curves following intraperitoneal injection of LPS on day 1.

FIG. 8. Kaplan Meier survivor curves following intraperitoneal injection of LPS on day 1.

### DETAILED DESCRIPTION

### Definitions

In the context of this specification, the terms “a” and “an” are used herein to refer to one or to more than one (ie, to at least one) of the Grammatical object of the article. By way of example, “an element” means one element or more than one element.

The term “about” is understood to refer to a range of +/−10%, preferably +/−5% or +/−1% or, more preferably, +/−0.1%.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and preferably together in the same formulation.

The terms “comprise”, “comprises”, “comprised” or “comprising”, “including” or “having” and the like in the present specification and claims are used in an inclusive sense, i.e., to specify the presence of the stated features but not preclude the presence of additional or further features.

The term “endotoxemia” refers to conditions associated with undesirable levels of endotoxin in the serum of a subject. Endotoxemia as used herein may be associated with detectable endotoxin levels of at least 5 pm/mL, at least 10 pg/mL, at least 20 pg/mL, at least 50 pg/mL or at least 100 pg/mL.

The term “pharmaceutically acceptable” as used herein refers to substances that do not cause substantial adverse allergic or immunological reactions when administered to a subject. A “pharmaceutically acceptable carrier” includes, but is not limited to, solvents, coatings, dispersion agents, wetting agents, isotonic and absorption delaying agents and disintegrants.

The term “prevention” includes reduction of risk, incidence and/or severity of an condition or disorder including a bacterial infection. The terms “treatment” and “treat” include both prophylactic or preventive treatment (that prevent and/or slow the development of a targeted pathologic condition or disorder including a bacterial infection) and curative, therapeutic or disease-modifying treatment, including therapeutic measures that cure, slow down, lessen symptoms of, and/or halt progression of a diagnosed pathologic condition or disorder; and treatment of patients at risk of contracting a particular condition or suspected to have contracted a particular condition, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a particular condition but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

### Haloperoxidase

Haloperoxidases are a class of peroxidase enzymes that catalyse the oxidation of halides in the presence of hydrogen peroxide. Mammalian haloperoxidases include myeloperoxidase, eosinophil peroxidase and lactoperoxidase. Myeloperoxidase (MPO) is a dimeric heme A glycoprotein produced by neutrophil and monocyte leukocytes. Eosinophil peroxidase (EPO) is produced by eosinophil leukocytes and has moderate homology to MPO (72.4% at the nucleotide and 69.8% at the amino acid level). Both MPO and EPO display potent microbicidal activities.

Due to the selective binding properties of haloperoxidases, when a target microorganism, such as a pathogenic microorganism, has a binding capacity for a haloperoxidase which is greater than that of a desired microorganism, such as members of the lactic acid bacteria which form part of the normal flora, the target microorganism is selectively bound by the haloperoxidase with little or no binding to the desired microorganism. In this regard, haloperoxidases are capable of selectively killing Gram-negative bacteria. In the presence of peroxide and halide, the target-bound haloperoxidase catalyzes halide oxidation and facilitates the disproportionation of peroxide to singlet molecular oxygen (¹O₂) at the surface of the target microorganism. Singlet molecular oxygen has a microsecond lifetime and a reactive radius of about 0.2 micrometers. As such, combustive microbicidal action may be somewhat limited to haloperoxidase-bound microbes with little collateral damage to beneficial microbes or host cells.

MPO binds to a broad spectrum of bacteria including all Gram-negative bacteria tested (eg, Pseudomonas aeruginosa and Escherichia coli), and many Gram-positive bacteria tested (eg, Staphylococcus aureus). As disclosed in U.S. Pat. Nos. 5,888,505, 6,294,168 and 8,945,540, MPO is able to kill target microorganisms in the presence of a peroxide and halide without significantly damaging surrounding tissue and normal flora. EPO also displays potent microbicidal activity.

Notwithstanding the known halide- and peroxide-dependent microbicidal activities of MPO and EPO, the present inventors have surprisingly found that MPO and EPO are able to bind to, and inhibit, endotoxin activity, including LPS and Lipid A activity, independently of their haloperoxidase enzymatic activity. As described herein, inhibition does not require hydrogen peroxide-dependent production of hypochlorite (or hypobromite), or secondary production of singlet molecular oxygen. As such, the composition of the present disclosure may comprise a haloperoxidase wherein the haloperoxidase is not engaged in active haloperoxidase activity and no peroxide-producing oxidase, thereby avoiding potential risks such as hypoglycemia associated with administering a peroxide-producing oxidase to a subject, particularly systemically. Accordingly, the present disclosure provides compositions and methods for inhibiting LPS and Lipid A activity comprising contacting the LPS or Lipid A with a haloperoxidase such as MPO or EPO wherein the haloperoxidase is not engaged in active haloperoxidase activity. The haloperoxidase may not engage in active haloperoxidase activity or may be rendered enzymatically inactive by avoiding its exposure to a defined substrate such as peroxide or a halide, or by modifying the haloperoxidase itself. For example, the haloperoxidase may comprise mutations such as amino acid substitutions, deletions or additions which render the haloperoxidase enzymatically inactive without compromising its ability to bind to, and inhibit, LPS or Lipid A.

Moreover, the present inventors have also surprisingly found that the interaction between each haloperoxidase and LPS or Lipid A is not controlled by electrostatic binding alone. As described herein, MPO is less cationic (i.e., more neutral) than EPO but may be more efficient at binding and inhibiting LPS, which is anionic. Being less cationic than EPO, MPO may be particularly suitable for systemic administration, and as such, the treatment of systemic conditions such as septicemia and sepsis.

### Compositions

The present disclosure provides compositions suitable for treating bacterial infections and endotoxin-related conditions. The compositions comprise a haloperoxidase, such as MPO or EPO, which are capable of antimicrobial action through oxidase-driven hydrogen peroxide production and haloperoxidase generation of hypochlorite and singlet molecular oxygen via the haloperoxidase's enzymatic activity. The enzymatic activity of MPO or EPO can also result in result in LPS and Lipid A inhibition, but this endotoxin inhibition does not require the production of hypochlorite or singlet molecular oxygen. In addition, the present inventors have also found that MPO and EPO are able to bind to, and inhibit, endotoxin activity, including LPS and Lipid A activity, independently of haloperoxidase enzymatic activity. Accordingly, the compositions of the present disclosure may produce no, or substantially no, singlet molecular oxygen when used to inhibit LPS or Lipid A activity.

The compositions of the present disclosure may comprise about 0.05 μg/mL or more of haloperoxidase (eg, MPO or EPO) such as at least about 1 μg/mL, about 2 μg/mL, about 5 μg/mL, about 10 μg/mL, about 20 μg/mL about 50 μg/mL, about 100 μg/mL, about 500 μg/mL or about 1,000 μg/mL. In some embodiments, the compositions of the present disclosure comprise from about 1 μg/mL to about 50,000 μg/mL of haloperoxidase (e.g., MPO or EPO), or from about 5 μg/mL to about 10,000 μg/mL of haloperoxidase, or from about 10 μg/mL to about 5,000 μg/mL of haloperoxidase. The composition may comprise the haloperoxidase at a concentration of less than about 50,000 μg/mL, such as less than about 20,000 μg/mL, less than about 10,000 μg/mL, less than about 5,000 μg/mL, less than about 1,000 μg/mL, less than about 500 μg/mL, less than 100 μg/mL or less than about 50 μg/mL. In other embodiments, the haloperoxidase (eg, MPO or EPO) is present in the composition at a concentration of about 6 μg/mL, 12 μg/mL, 25 μg/mL, 60 μg/mL or 120 μg/mL.

In certain embodiments, the present disclosure comprises administering a first composition comprising a haloperoxidase (e.g., MPO or EPO) to the site of infection; and administering a second composition comprising a substrate for the oxidase to the site of infection. In some embodiments, the first composition and the second composition are mixed before administration to the site of infection. In some embodiments, the first composition and the second composition are administered concurrently to the site of infection. In some embodiments, the first composition and the second composition are administered sequentially to the site of infection. The first composition and the second composition may be administered in any order.

In certain embodiments, the composition of the present disclosure further comprises a second antimicrobial agent. The second antimicrobial agent may contribute to killing target microbes which then release endotoxins. The damage caused by those endotoxins may be attenuated by the haloperoxidase (eg, MPO or EPO) which is also present in the composition. In some embodiments, the haloperoxidase may be conjugated to the second antimicrobial agent. In such embodiments, the haloperoxidase, which may lack its haloperoxidase enzymatic activity, selectively targets a pathogenic microrganism, such as a Gram-negative bacterium, and thus exposes it to the conjugated second antimicrobial agent. In other embodiments, the haloperoxidase (e.g., MPO or EPO) and the second antimicrobial agent are formulated such that they are administered simultaneously or sequentially. Suitable antimicrobial agents may include antimicrobial peptides, antimicrobial antibodies or fragments thereof, aminoglycoside, polyene, nitroimidazole, rifamycin, bacitracin, a beta-lactam, cephalosporin, chloramphenicol, a glycopeptide, a macrolide, a lincosamide, penicillin, a quinolones, rifampicin, tetracycline, trimethoprim a sulfonamide, amoxicillin, augmentin, amoxicillin, ampicillin, azlocillin, flucloxacillin, mezlocillin, methicillin, cephalexin, cefazedone, cefuroxime, loracarbef, cemetazole, cefotetan, cefoxitin, ciprofloxacin, levaquin, floxacin, doxycycline, minocycline, gentamycin, amikacin, tobramycin, clarithromycin, azithromycin, erythromycin, daptomycin, neomycin, kanamycin, streptomycin, nisin, epidermin, gallidennin, cinnamycin, duramycin, lacticin 481, amoxicillin, amoxicillin/clavulanic acid, metronidazole, clindamycine, chlortetracycline, dcmeclocycline, oxytetracycline, amikacin, netilmicin, cefadroxil, cefazolin, cephalexin, cephalothin, cephapirin, cephradine, cefaclor, cefamandole, cefametazole, cefonicid, cefotetan, cefoxitine, cefpodoxime, cefprozil, cefuroxime, cefdinir, cefixime, cefoperazone, cefotaxime, ceftazidime, ceftibuten, ceftizoxime, ceftriaxone, cefepime, azithromycin, claforan, clarithromycin, dirithromycin, erythromycin, lincomycin, troleandomycin, bacampicillin, carbenicillin, cloxacillin, dicloxacillin, meticillin, mezlocillin, nafcillin, oxacillin, piperacillin, ticarcillin, cinoxacin, ciprofloxacin, enoxacin, grepafloxacin, levofloxacin, lomefloxacin, nalidixic acid, norfloxacin, ofloxacin, sparfloxacin, sulfisoxazole, sulfacytine, sulfadiazine, sulfamethoxazole, sulfisoxazole, dapson, aztreonam, bacitracin, capreomycin, clofazimine, colistimethate, colistin, cycloserine, fosfomycin, furazolidone, methenamine, nitrofurantoin, pentamidine, rifabutin, spectinomycin, tigecycline, trimethoprim, trimetrexate glucuronate, vancomycin, chlorhexidine, carbapenem and ertapenem. The second antimicrobial agent may be selected from penicillins, cephalosporins, carbacephems, cephamycins, carbapenems, monobactams, aminoglycosides, glycopeptides, quinolones, tetracyclines, macrolides and fluoroquinolones.

The compositions of the present disclosure may further comprise a pharmaceutically acceptable carrier. In some embodiments, the compositions are provided in a liquid carrier. Any liquid carrier may generally be used for this purpose, provided that the carrier does not significantly interfere with the selective binding capabilities of the haloperoxidase or with enzyme activity (if enzymatically-catalysed microbicidal action is desired). Alternatively, the compositions may be provided in solid form with activation on solubilization in liquid.

In preferred embodiments, the compositions of the present disclosure are suitable for systemic administration. For example, the composition may be prepared as a liquid for direct application to the blood stream. The composition may be used in the form of a sterile aqueous solution which may contain other substances, for example, enough salts or solutes to make the solution isotonic with blood. The aqueous solutions should be suitably buffered (preferably to a pH of from about 3 to 9), if appropriate. In preferred embodiments, the compositions described herein are suitable for parenteral administration, for example intravenous administration and/or topical application. The preparation of suitable parenteral compositions under sterile conditions is readily accomplished by standard pharmaceutical techniques well known to those skilled in the art. Compositions suitable for parenteral administration may include aqueous and non-aqueous sterile injection solutions which may contain anti-oxidants, buffers and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets.

Compositions for parenteral administration include aqueous solutions. Additionally, suspensions may be prepared as appropriate oily injection suspensions. Suitable lipophilic solvents or vehicles include fatty oils such as sesame oil or synthetic fatty acid esters such as ethyl oleate or triglycerides. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

In certain embodiments, the present disclosure provides a composition suitable for treating septicemia, endotoxemia, a bacterial infection, or a Gram-negative bacterial infection in a subject, the composition comprising: a therapeutically effective amount of haloperoxidase, such as myeloperoxidase or eosinophil peroxidase; and a pharmaceutically acceptable carrier, wherein the composition is suitable for systemic administration to the subject.

In certain embodiments, the present disclosure provides a composition suitable for treating septicemia, endotoxemia, a bacterial infection, or a Gram-negative bacterial infection in a subject, the composition comprising: a therapeutically effective amount of haloperoxidase, such as myeloperoxidase or eosinophil peroxidase; and a pharmaceutically acceptable carrier, wherein the composition is suitable for parenteral administration to the subject.

In certain embodiments, the present disclosure provides a composition suitable for treating septicemia, endotoxemia, a bacterial infection, or a Gram-negative bacterial infection in a subject the composition comprising: a therapeutically effective amount of haloperoxidase, such as myeloperoxidase or eosinophil peroxidase; and a pharmaceutically acceptable carrier, wherein the composition is suitable for intravenous administration to the subject.

In certain embodiments, the present disclosure provides a composition suitable for treating septicemia, endotoxemia, a bacterial infection, or a Gram-negative bacterial infection in a subject, the composition comprising: less than about 1,000 μg/mL myeloperoxidase or eosinophil peroxidase; and a pharmaceutically acceptable carrier, wherein the composition is suitable for intravenous administration to the subject.

In certain embodiments, the present disclosure provides a composition suitable for treating a Gram-negative bacterial infection in a subject, the composition comprising: a therapeutically effective amount of myeloperoxidase or eosinophil peroxidase; and a pharmaceutically acceptable carrier, wherein the composition is suitable for topical administration to the site of the Gram-negative bacterial infection.

In certain embodiments, the present disclosure provides a composition suitable for treating a Gram-negative bacterial infection in a subject, the composition comprising a therapeutically effective amount of a haloperoxidase and a pharmaceutically acceptable carrier, wherein the haloperoxidase is not engaged in active haloperoxidase activity, but available for LPS or Lipid A binding and inhibition.

In certain embodiments, the present disclosure provides a composition suitable for treating septicemia in a subject, the composition comprising a therapeutically effective amount of a haloperoxidase and a pharmaceutically acceptable carrier, wherein the haloperoxidase is not engaged in active haloperoxidase activity, but available for LPS or Lipid A binding and inhibition.

In certain embodiments, the present disclosure provides a composition suitable for treating endotoxemia in a subject, the composition comprising a therapeutically effective amount of a haloperoxidase and a pharmaceutically acceptable carrier, wherein the haloperoxidase is not engaged in active haloperoxidase activity, but available for LPS or Lipid A binding and inhibition.

In certain embodiments, the present disclosure provides a composition suitable for treating a bacterial infection in a subject the composition comprising a therapeutically effective amount of a haloperoxidase and a pharmaceutically acceptable carrier, wherein the haloperoxidase is not engaged in active haloperoxidase activity, but available for LPS or Lipid A binding and inhibition.

The compositions of the present disclosure may be suitable for the topical treatment of microbial infections in a human or non-human mammalian subject at sites permitting direct contact of the compositions of the present disclosure with the microbial infection, such as, for example, infections of the skin, eyes, ears, mouth, nasal and sinus passages, traumatic injury sites, surgical sites and the like.

In certain embodiments, the composition is administered intranasally or by inhalation (for example, in the form of an aerosol spray presentation from a pressurised container, pump, spray or nebuliser with the use of a suitable propellant, such as dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas). In the case of a pressurised aerosol, the dosage unit may be determined by providing a valve to deliver a metered amount. The pressurised container, pump, spray or nebuliser may contain a solution or suspension of the haloperoxidase, e.g., using a mixture of ethanol and the propellant as the solvent, which may additionally contain a lubricant, e.g., sorbitan trioleate.

Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including topical application, intramuscular, subcutaneous, transcutaneous, intradermal, intramedullary delivery (e.g., injection), as well as intrathecal, direct intraventricular, intravenous, intraperitoneal, intranasal, or intraocular delivery (e.g., injection). For injection, the haloperoxidase may be formulated in an aqueous solution, suitably in physiologically compatible buffers such as Hanks' solution, Ringer's solution, or physiological saline buffer. For topical administration, penetrants appropriate to the barrier to be permeated may be used in the formulation. Such penetrants are generally known in the art.

Alternatively, the haloperoxidase may be formulated using pharmaceutically acceptable carriers well known in the art into dosages suitable for oral administration such as tablets, pills, capsules, liquids, gels, syrups, slurries, suspensions and the like. Suitable carriers may be selected from malt, gelatine, talc, calcium sulphate, vegetable oils, synthetic oils, polyols, alginic acid, phosphate buffered solutions, emulsifiers, isotonic saline, and pyrogen-free water.

### Methods

The present disclosure provides methods for treating bacterial infections and endotoxin-related conditions. In preferred embodiments, the methods and compositions of the present disclosure are suitable for treating systemic infections such as septicemia, which is a condition that arises when bacteria or endotoxins enter the bloodstream. Septicemia can be triggered by an infection in one part of the body, such as a urinary tract infection, an abdominal infection, a lung infection (eg, pneumonia) or a kidney infection, which then spreads to other parts of the body. The compositions of the present disclosure are, in preferred embodiments, suitable for systemic administration and therefore need not be administered directly to the site of initial infection. Conditions associated with septicemia include sepsis, organ dysfunction, acute respiratory distress syndrome, systemic inflammatory response syndrome and septic shock.

In certain embodiments, the present disclosure provides a method of treating a bacterial infection in a subject, the method comprising administering to the subject a composition comprising a therapeutically effective amount of a haloperoxidase as described herein. The bacterial infection may be a Gram-positive or a Gram-negative bacterial infection. Haloperoxidase enzymatic action kills Gram-positive and Gram-negative bacteria, but haloperoxidase, in the absence of enzymatic action, may only inhibit endotoxin activity of Gram-negative bacteria. Preferably, the bacterial infection is a Gram-negative bacterial infection such as an infection caused by Treponema spp., Borrelia spp., Citrobacter freundii, Enterobacter cloacae, Escherichia coli, Klebsiella pneumoniae, Proteus mirabilis, Acinetobacter spp., Pseudomonas aeruginosa, Aeromonas hydrophila or Pasteurella multocida. In certain embodiments, the bacterial infection is a Gram-negative bacterial infection such as an infection caused by a species of Enterobacteriaceae (eg, a species in Klebsiella, Enterobacter, Shigella, Escherichia, Salmonella, Yersinia, or Citrobacter), a species of Pseudomonas, a species of Acinetobacter or any combination thereof. In other embodiments, the infection is caused by Klebsiella pneumonia, Enterobacter cancerogenus, Enterobacter cloacae, Enterobacter hormaechei, Enterobacter asburiae, Shigella boydii, Shigella dysenteriae, Shigella flexneri, Shigella sonnei, Escherichia coli, Escherichia fergusonii, Salmonella choleraesuis, Salmonella choleraesuis subsp. indica, Salmonella enteritidis, Salmonella virchow, Salmonella paratyphi B, Salmonella typhimurium, Salmonella paratyphi A, Salmonella typhi, Salmonella choleraesuis subsp. arizonae, Salmonella choleraesuis subsp. diarizonae, Salmonella choleraesuis subsp. houtenae, Salmonella bongori, Citrobacter sedlakii, Citrobacter braakii, Citrobacter werkmanii, Citrobacter freundii, Citrobacter youngae, Citrobacter amalonaticus, Yersinia enterocolitica, Yersinia frederiksenii, Yersinia pestis, Yersinia pseudotuberculosis, or any combination thereof. In addition, the compositions of the present disclosure may be useful for killing Gram-positive bacteria such as Bacillus sps. and Clostridium sps., as well as inhibiting the growth of, or killing, spore forming microorganisms and fungi such as Aspergillus sps., Fusarium sps., and Trichophyton sps.

Also provided herein are methods of treating endotoxemia in a subject the method comprising administering to the subject a composition comprising a therapeutically effective amount of a haloperoxidase as described herein. The endotoxemia may be associated with sepsis, toxic shock, fever, diarrhea, septic shock, gastroenteritis, pneumonia, meningitis, endocarditis, osteomyelitis, cholecystitis, cholangitis, meningitis, enteric fever, shigellosis, multiple organ dysfunction syndrome, peritonitis, neutropenia, urosepsis, liver dysfunction, kidney dysfunction, pancreatitis, leaky bowel syndrome, menigingococcemia or systemic inflammatory response syndrome.

Endotoxemia generally refers to conditions associated with undesirable levels of endotoxin in the serum of a subject. Early symptoms of endotoxemia can include fever, redness and swelling. Endotoxin concentrations can vary widely in individuals depending on their health status, the presence of stresses such as intestinal hypoxia or hemorrhagic shock, acute conditions such as pancreatitis, chronic conditions such as periodontitis, the presence of infections and liver competency. Otherwise healthy individuals have been observed with transient mild endotoxemia (eg, blood endotoxin concentrations of 5 to 15 pg/mL) after physical stress such as prolonged athletic competition (Camus et al. Clin. Sci. (Lond.) 1997. 92, 415-422; Jeukendrup et al. Clin. Sci. (Lond.) 2000. 98, 47-55). In patients suffering from more severe cases of endotoxemia, such as patients suffering from sepsis or septic shock, endotoxin levels can be several fold higher (Venet et al. Intensive Care Med. 2000. 26, 538-544; Marshall et al. Crit. Care. 2002. 6, 289-290).

Patients suffering from endotoxemia are often at risk of developing related conditions. For example, patients suffering from endotoxemia are at increased risk of developing diabetes (Pussinen et al. Diabet. Care. 2011. 24, 392-397). In treating such patients, administration of a formulation comprising glucose oxidase may be undesirable as doing so may trigger or accelerate hypoglycemia in the patient and may be lethal. In that regard, the compositions of the present disclosure comprise a haloperoxidase (eg, MPO or EPO) and are suitable for treating endotoxemia. However, the haloperoxidase need not be enzymatically active, and as such, the compositions need not include glucose or a glucose oxidase.

The compositions of the present disclosure may be used to treat a number of endotoxin-mediated disorders and conditions associated with bacterial infection. For example, the compositions may be used to treat pneumonia, a urinary tract infection (UTI), septicemia, meningitis, diarrhea, a soft tissue infection, a skin infection, bacteremia, a respiratory system infection, endocarditis, an intra-abdominal infection, septic arthritis, osteomyelitis, a CNS infection, an ophthalmic infection, cholecystitis, cholangitis, meningitis, typhoid fever, food poisoning, gastroenteritis, enteric fever, shigellosis, a blood stream infection, intra-abdominal sepsis, a brain abscess, sepsis, a joint infection, a bone infection, a gastrointestinal infection or a wound infection.

The compositions of the present disclosure may be administered by a variety of routes. Preferably, the composition is administered systemically such as by direct delivery into the bloodstream of a subject. In certain embodiments, the composition is delivered parenterally. Exemplary routes of parenteral administration include, but are not limited to, intravascular, intracapsular, intraorbital, intracardiac, intradermal, transtacheal, intraperitoneal, intraventricular, intracerebroventricular, intrathecal, subcutaneous, subcuticular, subcapsular, subarachnoid, intraspinal, epidural, intrasternal, intracranial, intramuscular, intraarticular, intra-arterial, intranodal and intravenous. In one example, the composition is delivered intra-arterially, into an aorta, into an atrium or ventricle of the heart or into a blood vessel, eg, intravenously. In preferred embodiments, the composition is administered intraperitoneally or intravenously. In certain embodiments, the preferred route of administration is topical, wherein the composition is administered to a particular location such as a localized area of the body or to the surface of a body part, e.g. an infection site or a wound.

In certain embodiments, the present disclosure provides a method of treating septicemia in a human subject, the method comprising administering to the subject a composition comprising a therapeutically effective amount of myeloperoxidase or eosinophil peroxidase, wherein the composition is administered systemically to the subject.

In certain embodiments, the present disclosure provides a method of treating septicemia in a human subject, the method comprising administering to the subject a composition comprising a therapeutically effective amount of myeloperoxidase or eosinophil peroxidase, wherein the composition is administered parenterally to the subject.

In certain embodiments, the present disclosure provides a method of treating septicemia in a human subject, the method comprising administering to the subject a composition comprising a therapeutically effective amount of myeloperoxidase, wherein the composition is administered intravenously to the subject.

In certain embodiments, the present disclosure provides a method of treating septicemia in a human subject, the method comprising administering to the subject a composition comprising less than about 1,000 μg/mL myeloperoxidase or eosinophil peroxidase, wherein the composition is administered intravenously to the subject.

In certain embodiments, the present disclosure provides a method of treating endotoxemia in a human subject, the method comprising administering to the subject a composition comprising a therapeutically effective amount of myeloperoxidase or eosinophil peroxidase, wherein the composition is administered systemically to the subject.

In certain embodiments, the present disclosure provides a method of treating endotoxemia in a human subject, the method comprising administering to the subject a composition comprising a therapeutically effective amount of myeloperoxidase or eosinophil peroxidase, wherein the composition is administered parenterally to the subject.

In certain embodiments, the present disclosure provides a method of treating endotoxemia in a human subject, the method comprising administering to the subject a composition comprising a therapeutically effective amount of myeloperoxidase or eosinophil peroxidase, wherein the composition is administered intravenously to the subject.

In certain embodiments, the present disclosure provides a method of treating endotoxemia in a human subject, the method comprising administering to the subject a composition comprising less than about 1,000 μg/mL myeloperoxidase or eosinophil peroxidase, wherein the composition is administered intravenously to the subject.

In certain embodiments, the present disclosure provides a method of treating a bacterial infection in a human subject, the method comprising administering to the subject a composition comprising a therapeutically effective amount of myeloperoxidase or eosinophil peroxidase, wherein the composition is administered systemically to the subject.

In certain embodiments, the present disclosure provides a method of treating a bacterial infection in a human subject, the method comprising administering to the subject a composition comprising a therapeutically effective amount of myeloperoxidase or eosinophil peroxidase, wherein the composition is administered parenterally to the subject.

In certain embodiments, the present disclosure provides a method of treating a bacterial infection in a human subject, the method comprising administering to the subject a composition comprising a therapeutically effective amount of myeloperoxidase or eosinophil peroxidase, wherein the composition is administered intravenously to the subject.

In certain embodiments, the present disclosure provides a method of treating a bacterial infection in a human subject, the method comprising administering to the subject a composition comprising: less than about 1,000 μg/mL myeloperoxidase or eosinophil peroxidase, wherein the composition is administered intravenously to the subject.

In certain embodiments, the present disclosure provides a method of treating a Gram-negative bacterial infection in a human subject, the method comprising administering to the subject a composition comprising a therapeutically effective amount of myeloperoxidase or eosinophil peroxidase, wherein the composition is administered systemically to the subject.

In certain embodiments, the present disclosure provides a method of treating a Gram-negative bacterial infection in a human subject, the method comprising administering to the subject a composition comprising a therapeutically effective amount of myeloperoxidase or eosinophil peroxidase, wherein the composition is administered parenterally to the subject.

In certain embodiments, the present disclosure provides a method of treating a Gram-negative bacterial infection in a human subject, the method comprising administering to the subject a composition comprising a therapeutically effective amount of myeloperoxidase or eosinophil peroxidase, wherein the composition is administered intravenously to the subject.

In certain embodiments, the present disclosure provides a method of treating a bacterial infection in a human subject, the method comprising administering to the subject a composition comprising: less than about 1,000 μg/mL myeloperoxidase or eosinophil peroxidase, wherein the composition is administered intravenously to the subject.

In certain embodiments, the present disclosure provides a method of treating a Gram-negative bacterial infection in a human subject, the method comprising administering to the site of the Gram-negative bacterial infection in the subject a composition comprising a therapeutically effective amount of myeloperoxidase or eosinophil peroxidase.

In certain embodiments, the present disclosure provides a method of treating septicemia in a subject, the method comprising administering to the subject a composition comprising a therapeutically effective amount of a haloperoxidase, wherein the haloperoxidase is not engaged in active haloperoxidase activity, but available for LPS or Lipid A binding and inhibition.

In certain embodiments, the present disclosure provides a method of treating endotoxemia in a subject, the method comprising administering to the subject a composition comprising a therapeutically effective amount of a haloperoxidase, wherein the haloperoxidase is not engaged in active haloperoxidase activity, but available for LPS or Lipid A binding and inhibition.

In certain embodiments, the present disclosure provides a method of treating a bacterial infection in a subject, the method comprising administering to the subject a composition comprising a therapeutically effective amount of a haloperoxidase, wherein the haloperoxidase is not engaged in active haloperoxidase activity, but available for LPS or Lipid A binding and inhibition.

In certain embodiments, the present disclosure provides a method of treating a Gram-negative bacterial infection in a subject, the method comprising administering to the subject a composition comprising a therapeutically effective amount of a haloperoxidase, wherein the haloperoxidase is not engaged in active haloperoxidase activity, but available for LPS or Lipid A binding and inhibition.

In certain embodiments, the present disclosure provides a method of treating a Gram-negative bacterial infection in a human subject, the method comprising administering to the site of the Gram-negative bacterial infection in the subject a first composition comprising a therapeutically effective amount of myeloperoxidase or eosinophil peroxidase; and administering to the site of the bacterial infection in the subject a second composition comprising a substrate for the oxidase; wherein the first composition acts in combination with the second composition to kill the microbe and, in addition, inactivate a lipopolysaccharide endotoxin produced by the Gram-negative bacteria.

Dosages may vary with the type and severity of the condition to be treated, and may include single or multiple dosses. Specific dosage regimens may be adjusted over time according to the individual need and the professional judgment of the practitioner administering the composition. When administered to a human subject, the dosage regimen may vary depending on a variety of factors including the type and severity of infection or condition, the age, sex, weight or medical condition of the subject and the route of administration. In that regard, the precise amount of the haloperoxidase composition that is to be administered may depend on the judgement of the practitioner. The compositions described herein may be administered over a period of hours, days, weeks, or months, depending on several factors, including the severity of the infection or condition being treated, whether a recurrence is considered likely, etc. The administration may be constant, e.g., constant infusion over a period of hours, days, weeks, months, etc. Alternatively, the administration may be intermittent, eg, once per day over a period of days, once per hour over a period of hours, or any other such schedule as deemed suitable. In some embodiments, the composition of the present disclosure is administered once daily for at least one week, for example, at least once daily for at least two weeks, or once daily for at least one month or longer.

### EXAMPLES

### Example 1: Limulus Amebocyte Lysate Assay

Lipopolysaccharide (LPS) and Lipid A were quantified using the chromogenic Limulus amebocyte lysate (LAL) assay (LAL Endochrome-K, Charles River Endosafe). In brief, the LAL assay is a kinetic colorimetric assay that detects and measures the onset of color. Endotoxin activation of the LAL clotting enzyme is measured as enzymatic (hydrolytic) cleavage of a synthetic chromogenic substrate, releasing p-nitroaniline (pNA) which can be quantified by absorbance in a microplate spectrophotometer at a wavelength of 405 nm. Kinetic measurement at the time to maximum color change (slope) was used to gauge the activity of endotoxin present. Calibration standards were measured to generate a standard curve and equation for calculating the endotoxin activity of an assay sample. The amount of endotoxin present in a sample can be designated as endotoxin units (EU).

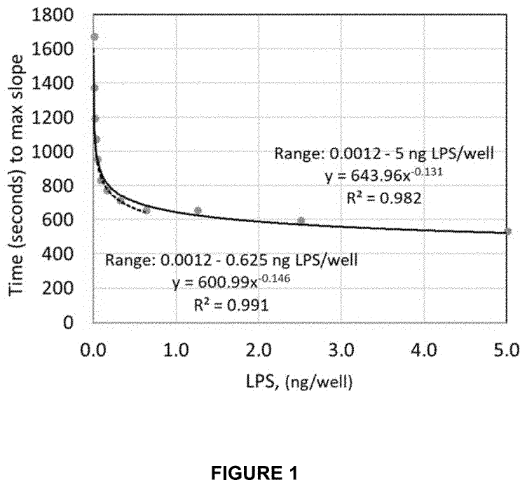

The endotoxin mass/well for a series of standards was plotted against the time to maximum color change (max slope, i.e., max velocity) (FIG. 1). The time limit for detection was 1680 seconds. The manufacturer (Sigma-Aldrich) specification for LPS activity was 3×10⁶ EU per mg (i.e., 3 EU/ng). For the 0.01 ng LPS/well standard, the time to maximum slope was 1200 seconds, a value equivalent to about 0.03 EU. This is consistent with a 0.05 EU/mL value for time to maximum slope time of 1400 seconds reported for the chromogenic LAL value assay (Charles River Endosafe).

### Example 2: Inhibition of LPS by Haloperoxidase

MPO, EPO or GO were pre-incubated with LPS or Lipid A for a period of 30 minutes at 37° C. Following pre-incubation, limulus lysate solution was added, and the seconds to maximum color change (slope) was measured. Using the LPS standards-derived equation (FIG. 1), the time to maximum slope was converted to equivalent LPS mass, and inhibition was calculated as the difference between the activity of the expected mass (e.g, endotoxin without enzyme) and the actual measured LPS activity (e.g., endotoxin with enzyme) expressed as a percentage.

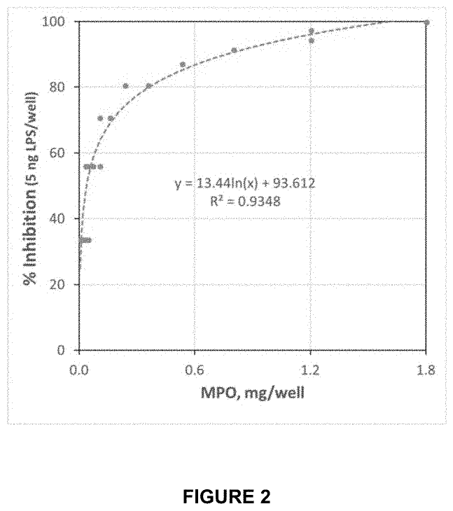

As illustrated in FIG. 2, MPO clearly inhibited LPS, with the inhibition of LPS being proportional to the natural log (Ln) of MPO mass. One mg of MPO inhibited 90.8% of the activity of 5 ng LPS (i.e., 4.5 ng LPS). 0.01 mg of MPO inhibited 27.7% of the activity of 5 ng LPS (i.e., 1.4 ng LPS). With the LPS mass held constant and MPO mass varied, the percent inhibition of LAL activity was directly proportional to the log of MPO mass. As the ratio of LPS to MPO mass increased, the percent inhibition of endotoxin activity decreased, but the efficiency of MPO inhibition increased, e.g., 0.003 mg (3 μg) MPO produced 11.2% inhibition of 5 ng LPS, i.e., 0.56 ng LPS.

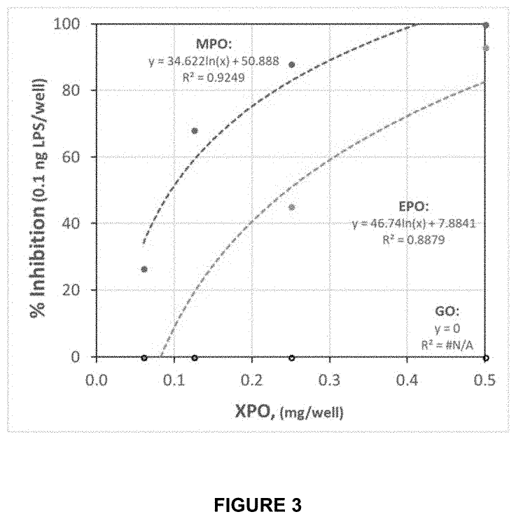

Similar inhibition assays were performed to determine the endotoxin inhibition activities of EPO and GO in addition to MPO. Referring to FIG. 3, both MPO and EPO inhibited LPS activity, and for a constant mass of LPS, the inhibition of endotoxin activity was proportional to the log of EPO and MPO mass. 2.5 mg of MPO and EPO produced 88% and 45.1% inhibition of 0.1 ng LPS activity, respectively; and 1.25 mg of MPO and EPO produced 68.1% and 0% inhibition, respectively. Glucose oxidase did not inhibit LPS within the range of concentrations tested.

MPO and, to a greater extent EPO, are cationic proteins. Electrostatic interactions may therefore contribute to MPO and EPO binding and inhibition of the anionic LPS as well as its Lipid A component. However, the surprising observation that MPO can inhibit LPS more efficiently than EPO, despite MPO being less cationic than EPO, suggests that the interaction between each haloperoxidase and LPS is likely to be controlled by more than electrostatic binding alone.

### Example 3: Inhibition of Lipid a Endotoxin Activity by MPO and EPO

Lipid A, the toxic component of LPS, is made up of two glucosamine units attached to phosphate groups and typically six hydrophobic fatty acids which anchor it to the outer membrane of Gram-negative bacteria. Purified Lipid A is toxic and its endotoxin activity is measurable by the LAL assay.

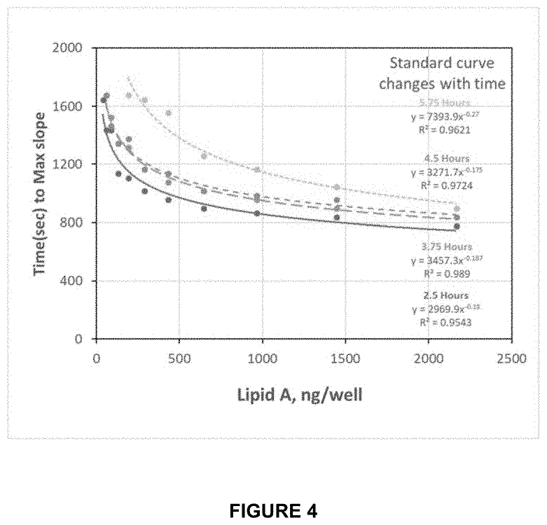

The inhibitory action of MPO and EPO was tested using diphosphoryl Lipid A from E. coli F583 (Sigma Aldrich, L5399) and the chromogenic LAL assay described above. The hydrophobic character of Lipid A can complicate preparation of aqueous standards. The data presented in FIG. 4 illustrates the reproducibility of aqueous Lipid A standards measured as chromogenic LAL activities up to 5.75 hours post preparation. The endotoxin unit activity per mass of Lipid A is lower than that described by the manufacturer (i.e., ≥1×10⁶ EU/mg) using a coagulation LAL assay, and activities showed detectable degradation over several hours. As such, LPS or Lipid A standards were simultaneously run and used to calculate a standard Lipid A curve and equation for each MPO or EPO inhibition experiment.

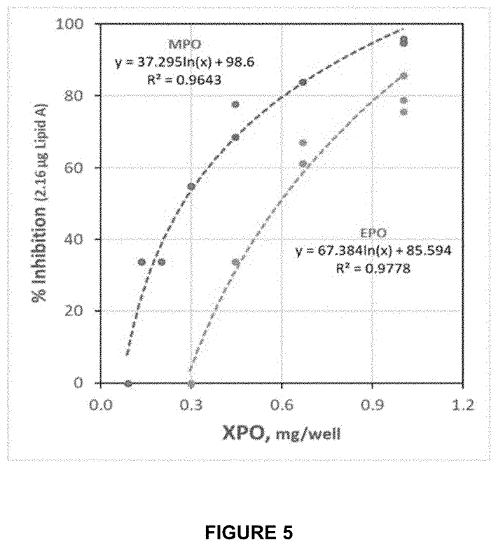

Both MPO and EPO were clearly able to inhibit Lipid A activity with MPO being more inhibitory than EPO (FIG. 5). As was the case for LPS, Lipid A inhibition was proportional to the log of MPO and EPO mass. GO did not inhibit the endotoxin activity of Lipid A.

### Example 4: Effect of Haloperoxidase Enzymatic Activity on Endotoxin Inhibition

The experiments described in Examples 2 and 3 were conducted in the absence of peroxide or a peroxide-generating system. As such, the experiments were conducted in the absence of haloperoxidase enzymatic activity. To determine whether haloperoxidase enzymatic activity affected the extent of endotoxin inhibition, a formulation comprising 4 μg MPO and 0.8 μg GO with chloride was tested for LPS inhibitory activity in the absence of D-glucose (no haloperoxidase activity) and in the presence of D-glucose (high haloperoxidase activity).

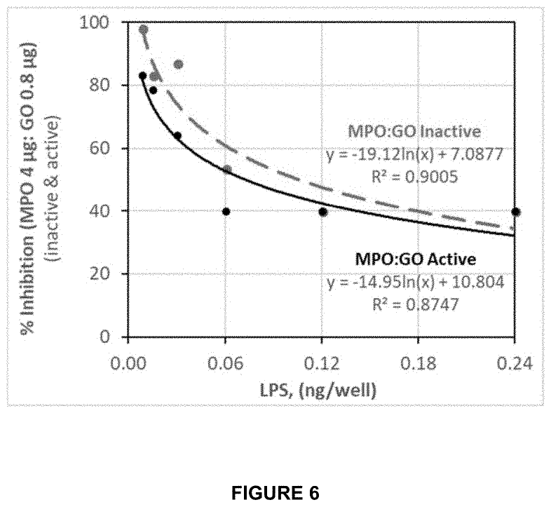

With MPO mass held constant and LPS mass varied, the percent inhibition of endotoxin activity was inversely proportional to the log of LPS mass; the opposite relationship to that observed when LPS or Lipid A are held constant and MPO or EPO are varied (FIG. 6). As the ratio of LPS to MPO mass increases, the percent inhibition of endotoxin activity decreases. The endotoxin inhibitory activities of the enzymatically active and inactive MPO formulation are essentially equivalent. The results demonstrate that haloperoxidase activity does not significantly effect endotoxin inhibition.

### Example 5: Halo Peroxidase-Mediated Protection from Endotoxin Toxicity

Experimentally naïve, healthy BALB/c female mice with a weight range of 16.2 to 19.7 g were divided into 4 dose groups. Each group of 20 mice received a dose of purified LPS from E. coli 055:65 (Sigma-Aldrich L4524) injected intraperitoneally in a 0.5 mL volume of low endotoxin reagent water (LRW). Treatment of the animals (including but not limited to all husbandry, housing, environmental and feeding conditions) was conducted in accordance with the guidelines recommended in Guide for the Care and Use of Laboratory Animals. The results are presented in Table 1. The LD₉₀ was estimated to be 400 μg per mouse, ie, about 22 mg/kg.

TABLE 1
Establishment of lethal dose 90 (LD₉₀).

| | LPS | Dose/ | Total % | Total % | |
|---|---|---|---|---|---|
| Dose | Dose | mouse | Mortality | Mortality | Total % |
| Group* | (μg)** | (mg/kg)*** | Day 2 | Day 3 | Mortality |
| 1 | 200 | 11.0 | 20% | 25% | 25% |
| 2 | 350 | 19.3 | 45% | 75% | 75% |
| 3 | 500 | 27.6 | 100% | 100% | 100% |
| 4 | 650 | 35.9 | 100% | 100% | 100% |

*Twenty female mice per group.
**Dose volume 0.5 mL delivered as a single intraperitoneal injection of LPS.
***Calculated doses based on group mean body weight.

The effect of MPO on LPS activity was tested at the LD_{N} dose and at twice the LD_{N} dose. The appropriate concentrations of LPS and MPO were mixed and vortexed vigorously for approximately one minute. The samples were then incubated at 37° C. for 45 min, and vortexed again prior to intraperitoneal injection.

TABLE 2
Effect of MPO on LPS tolerance.

| | | | | Intraperitoneal |
|---|---|---|---|---|
| | | | | volume of |
| | | | | LPS and MPO |
| | Mice | LPS Dose | Myeloperoxidase | injected |
| Group | (female) | Level | (MPO) Dose | (mL) |
| A | 20 | None | 500 μg | 0.5 |
| B | 15 | 1x LD₉₀ | None | 0.5 |
| | | 400 μg/mouse | | |
| C | 20 | 1x LD₉₀ | 500 μg | 0.5 |
| | | 400 μg/mouse | | |
| D | 20 | 2x LD₉₀ | 500 μg | 0.5 |
| | | 800 μg/mouse | | |

*Dose level compared to LD₉₀ determined by toxicities observed in Phase 1.

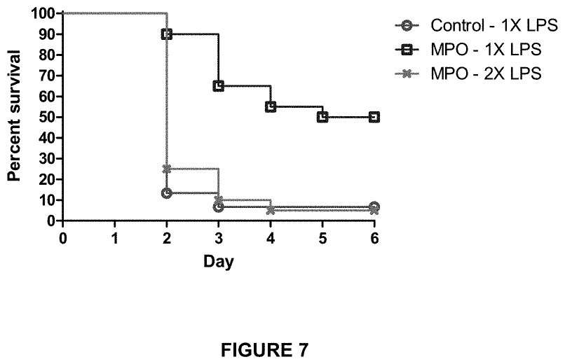

The porcine MPO employed was 98.9% pure by reverse phase ultra performance liquid chromatography (RP-UPLC), 100% pure by molecular size exclusion high performance liquid chromatography (SEC-HPLC) and had a reinheitszahl (RZ; A_{430 nm}/A_{280 nm}) of 0.79. Animals treated with 500 μg MPO without LPS displayed no abnormal clinical observations or mortality. In animals treated with MPO and LPS, the LPS-associated mortality clearly decreased. Mortality in 1×LPS/MPO-treated animals was 50%, and in 2×LPS/MPO-treated animals, was 95%. Kaplan Meier curves show both a decrease in mortality and a delay in time to mortality associated with MPO treatment (FIG. 7).

Testing was extended to include higher doses of MPO as well as EPO as set out in Table 3. The same purity of MPO was dosed at higher concentrations. The porcine EPO employed was 99.2% pure by reverse phase high performance liquid chromatography (RP-HPLC) and had a reinheitszahl (RZ; A_{415 nm}/A_{280 nm}) of 0.96.

TABLE 3
Effect of MPO and EPO on LPS tolerance.

| | | Additive Dose | Additive | Additive | Mice | |
|---|---|---|---|---|---|---|
| Group | LPS Conc. | (MPO or EPO) | MPO | EPO | (female) | Comment |
| E | 0 | 5000 μg/mouse | Vial #1 | — | 20 | MPO Only |
| | | | (MPO) | | | Control |
| F | 0 | 5000 μg/mouse | — | Vial #2 | 20 | EPO Only |
| | | | | (EPO) | | Control |
| G | 1x LD₉₀ of | 0 | Vial #3 (LPS) | | 20 | LPS Only |
| | 400 μg/mouse | | | | | Control |
| H | 1x LD₉₀ of | 5000 μg/mouse | Vial #4 | — | 20 | LPS + MPO |
| | 400 μg/mouse | | (LPS + MPO) | | | High Dose |
| I | 1x LD₉₀ of | 5000 μg/mouse | — | Vial #5 | 20 | LPS + EPO |
| | 400 μg/mouse | | | (LPS + EPO) | | High Dose |
| J | 1x LD₉₀ of | 2500 μg/mouse | Vial #6 | — | 20 | LPS + MPO |
| | 400 μg/mouse | | (LPS + MPO) | | | Low Dose |
| K | 1x LD₉₀ of | 2500 μg/mouse | — | Vial #7 | 20 | LPS + EPO |
| | 400 μg/mouse | | | (LPS + EPO) | | Low Dose |

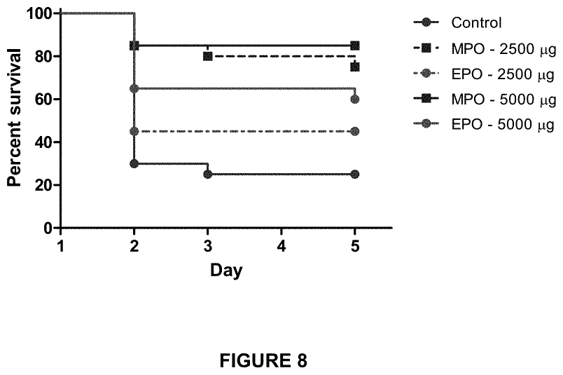

Animals treated with 5000 μg of MPO in the absence of LPS displayed no abnormal clinical observations or mortality. Animals treated with 5000 μg EPO in the absence of LPS also displayed no abnormal clinical observations, although two animals were found dead in their cages the day following intraperitoneal dosing for a mortality rate of 10%. The mortality rate of animals treated with 1×LPS (400 μg) in the absence of a haloperoxidase was 15 out of 20, or 75% (FIG. 8).

When 1×LPS (400 μg) was injected with MPO or EPO, the incidence of LPS-associated clinical observations decreased compared to LPS alone (FIG. 8). Endotoxin-associated mortality was clearly reduced in MPO- and EPO-treated animals at both 2500 and 5000 μg doses. MPO was more effective at reducing mortality than EPO, and for both MPO and EPO, the higher 5000 μg dose was more effective at reducing mortality compared to the 2500 μg dose.

It will be appreciated by those skilled in the art that the compositions and methods of the present disclosure may be embodied in many other forms.

1: A method of treating septicemia in a subject, the method comprising administering to the subject a composition comprising a therapeutically effective amount of a haloperoxidase, wherein the composition is administered systemically to the subject.

2: The method of claim 1 wherein the haloperoxidase is myeloperoxidase or eosinophil peroxidase.

3. (canceled)

4: The method of claim 2 wherein the composition is administered parenteraily to the subject.

5: The method of claim 3 wherein the composition is administered intravenously to the subject.

6: The method of any one of claims 1 to 5 wherein the subject is a human.

7: The method of any one of claims 1 to 6 wherein the haloperoxidase inhibits lipopolysaccharide (LPS) or Lipid A activity in the subject.

8: The method of any one of claims 1 to 6 wherein the haloperoxidase is not engaged in active haloperoxidase activity, but available for lipopolysaccharide (LPS) or Lipid A binding and inhibition.

9: The method of any one of claims 1 to 8 wherein the composition comprises the haloperoxidase at a concentration of less than about 50,000 μg/mL.

10: The method of claim 9 wherein the composition comprises the haloperoxidase at a concentration of less than about 1,000 μg/mL.

11: The method of any one of claims 1 to 10 wherein the haloperoxidase is myeloperoxidase.

12: The method of any one of claims 1 to 11 wherein the septicemia is associated with Gram-negative sepsis, organ dysfunction, acute respiratory distress syndrome, systemic inflammatory response syndrome or septic shock.

13: A method of treating endotoxemia in a subject, the method comprising administering to the subject a composition comprising a therapeutically effective amount of a haloperoxidase, wherein the composition is administered systemically to the subject.

14: The method of claim 13 wherein the haloperoxidase is myeloperoxidase or eosinophil peroxidase.

15. (canceled)

16: The method of claim 13 wherein the composition is administered parenterally to the subject.

17: The method of claim 16 wherein the composition is administered intravenously to the subject.

18: The method of any one of claims 13 to 17 wherein the endotoxemia is associated with a concentration of endotoxin in serum of the subject of greater than about 5 pg/mL.

19: The method of claim 18 wherein the endotoxemia is associated with a concentration of endotoxin in serum of the subject of greater than about 20 pg/mL.

20: The method of any one of claims 13 to 19 wherein the subject is a human.

21: The method of any one of claims 13 to 20 wherein the haloperoxidase inhibits lipopolysaccharide (LPS) or Lipid A activity in the subject.

22: The method of any one of claims 13 to 21 wherein the haloperoxidase is not engaged in active haloperoxidase activity, but available for lipopolysaccharide (LPS) or Lipid A binding and inhibition.

23: The method of any one of claims 13 to 22 wherein the composition comprises the haloperoxidase at a concentration of less than about 50,000 μg/mL.

24: The method of claim 23 wherein the composition comprises the haloperoxidase at a concentration of less than about 1,000 μg/mL.

25: The method of any one of claims 13 to 24 wherein the haloperoxidase is myeloperoxidase.

26: The method of any one of claims 13 to 25 wherein the endotoxemia is associated with septicemia, sepsis, toxic shock, fever, diarrhea, septic shock, gastroenteritis, pneumonia, meningitis, endocarditis, osteomyelitis, cholecystitis, cholangitis, enteric fever, shigellosis, multiple organ dysfunction syndrome, peritonitis, neutropenia, urosepsis, liver dysfunction, kidney dysfunction, pancreatitis, leaky bowel syndrome, menigingococcemia or systemic inflammatory response syndrome.

27: The method of claim 26 wherein the endotoxemia is associated with septicemia.

28: A method of treating a bacterial infection in a subject, the method comprising administering to the subject a composition comprising a therapeutically effective amount of a haloperoxidase, wherein the composition is administered systemically to the subject.

29: The method of claim 28 wherein the haloperoxidase is myeloperoxidase or eosinophil peroxidase.

30. (canceled)

31: The method of claim 29 wherein the composition is administered parenterally to the subject.

32: The method of claim 31 wherein the composition is administered intravenously to the subject.

33: The method of any one of claims 28 to 32 wherein the subject is a human.

34: The method of any one of claims 28 to 33 wherein the bacterial infection is a Gram-negative bacterial infection.

35: The method of any one of claims 28 to 34 wherein the haloperoxidase inhibits lipopolysaccharide (LPS) or Lipid A activity in the subject.

36: The method of any one of claims 28 to 35 wherein the haloperoxidase is not engaged in active haloperoxidase activity, but available for lipopolysaccharide (LPS) or Lipid A binding and inhibition.

37: The method of any one of claims 28 to 36 wherein the composition comprises the haloperoxidase at a concentration of less than about 50,000 μg/mL.

38: The method of claim 37 wherein the composition comprises the haloperoxidase at a concentration of less than about 1,000 μg/mL.

39: The method of any one of claims 28 to 38 wherein the haloperoxidase is myeloperoxidase.

40: The method of any one of claims 28 to 39 wherein the bacterial infection is caused by Treponema spp., Borrelia spp., Citrobacter freundii, Citrobacter Citrobacter braakii, Citrobacter werkmanii, Citrobacter youngae, Citrobacter amalonaticus, Enterobacter cloacae, Enterobacter cancerogenus, Enterobacter hormaechei, Enterobacter asburiae, Escherichia coli, Escherichia fergusonii, Klebsiella pneumoniae, Proteus mirabilis, Acinetobacter spp., Pseudomonas aeruginosa, Aeromonas hydrophila, Pasteurella multocida, Shigella boydii, Shigella dysenteriae, Shigella flexneri, Shigella sonnei, Salmonella choleraesuis, Salmonella choleraesuis subsp. indica, Salmonella enteritidis, Salmonella virchow, Salmonella paratyphi B, Salmonella typhimurium, Salmonella paratyphi A, Salmonella typhi, Salmonella choleraesuis subsp. arizonae, Salmonella choleraesuis subsp. diarizonae, Salmonella choleraesuis subsp. houtenae, Salmonella bongori, Yersinia enterocolitica, Yersinia frederiksenii, Yersinia pestis or Yersinia pseudotuberculosis.

41: A composition suitable for treating a Gram-negative bacterial infection in a subject, the composition comprising: a therapeutically effective amount of a haloperoxidase; and a pharmaceutically acceptable carrier, wherein the composition is suitable for systemic administration to the subject.

42: The composition of claim 41 wherein the haloperoxidase is myeloperoxidase or eosinophil peroxidase.

43: The composition of claim 41 or claim 42 wherein the composition is suitable for parenteral administration.

44: The composition of claim 43 wherein the composition is suitable for intravenous administration.

45: The composition of any one of claims 41 to 44 wherein the haloperoxidase inhibits lipopolysaccharide (LPS) or Lipid A activity in the subject.

46: The composition of any one of claims 41 to 45 wherein the haloperoxidase is not engaged in active haloperoxidase activity, but available for lipopolysaccharide (LPS) or Lipid A binding and inhibition.

47: The composition of any one of claims 41 to 46 wherein the composition comprises the haloperoxidase at a concentration of less than about 50,000 μg/mi.

48: The composition of claim 47 wherein the composition comprises the haloperoxidase at a concentration of less than about 1,000 μg/mL.

49: The composition of any one of claims 41 to 48 wherein the subject is a human.

50: The composition of any one of claims 41 to 49 wherein the haloperoxidase is myeloperoxidase.

51: A method of treating a Gram-negative bacterial infection in a subject, the method comprising administering to the site of the Gram-negative bacterial infection in the subject a composition comprising: a therapeutically effective amount of a haloperoxidase; wherein the haloperoxidase is selected from myeloperoxidase and eosinophil peroxidase and is not engaged in active haloperoxidase activity, but available for lipopolysaccharide (LPS) or Lipid A binding and inhibition.

52: The method of claim 51 wherein the haloperoxidase inhibits lipopolysaccharide (LPS) or Lipid A activity in the subject.

53. (canceled)

54: The method of any one of claims 51 to 52 wherein the composition comprises from about 1 μg/mL to about 50,000 μg/mL, of the haloperoxidase.

55: The method of claim 51 or claim 52 wherein the composition comprises from 10 μg/mL to 5,000 μg/mL of the haloperoxidase.

55: The method of any one of claims 51 to 54 wherein the subject is suffering from a bacterial infection of the gum, eye, ear, skin, soft tissue, wound, vaginal area, groin area, bed sore or burn area, and preferably the method is a non-systemic use for treatment of superficial infection or wound.

57: The method of any one of claims 51 to 56 wherein the haloperoxidase is eosinophil peroxidase.

58: The method of any one of claims 51 to 57 wherein the subject is a human.

59: A method of treating a Gram-negative bacterial infection in a subject the method comprising administering to the site of the Gram-negative bacterial infection in the subject a first composition comprising a therapeutically effective amount of a haloperoxidase, wherein the haloperoxidase is not engaged in active haloperoxidase activity, but available for lipopolysaccharide (LPS) or Lipid A binding and inhibition; and administering to the site of the bacterial infection in the subject a second composition comprising a substrate for the oxidase; wherein the first composition acts in combination with the second composition to kill the microbe and inactivate a lipopolysaccharide (LPS) endotoxin produced by the Gram-negative bacteria.

60: The method of claim 59 wherein the haloperoxidase is myeloperoxidase or eosinophil peroxidase.

61: The method of claim 59 or claim 60 wherein the first composition and the second composition are mixed before administration to the site of infection.

62: The method of claim 59 or claim 60 wherein the first composition and the second composition are administered concurrently to the site of infection.

63: The method of claim 59 or claim 60 wherein the first composition and the second composition are administered sequentially to the site of infection.

64: The method of any one of claims 59 to 63 wherein the haloperoxidase is eosinophil peroxidase.

65: The method of any one of claims 59 to 64 wherein the subject is a human.
